(19) United States
(12) Patent Application Publication
(10) Pub. No.: US 2026/0259209 A1
(43) Pub. Date: Sep. 3, 2026

(54) TARGETING ABERRANT O-GLYCANS OF IGA1 TO DEVELOP A URINARY DIAGNOSTIC METHOD FOR IGA NEPHROPATHY

(71) Applicant: Buddhist Tzu Chi Medical Foundation, Hualien City (TW)

(72) Inventors: Ann CHEN, Hualien City (TW); Shuk-Man Ka, Hualien City (TW)

(21) Appl. No.: 19/407,614

(22) Filed: Dec. 3, 2025

(30) Foreign Application Priority Data
Mar. 2, 2025 (TW) 114107558

(51) Int. Cl. G01N 33/564; G01N 33/68

(52) U.S. Cl. CPC G01N 33/564; G01N 33/686; G01N 2440/38; G01N 2800/347

(57) ABSTRACT

The current invention provides high specificity monoclonal antibodies or lectins as probes, which can specifically bind to Gd-IgA as a novel non-invasive method for rapid detecting of urinary samples from IgAN subjects, which can elevate effectively to diagnosing IgA nephropathy.

### CROSS-REFERENCE TO RELATED APPLICATION

This application claims priority to TW application No. 114107558, filed Mar. 2, 2025, the contents of which are incorporated herein in their entirety.

### REFERENCE TO AN ELECTRONIC SEQUENCE LISTING

The application contains a Sequence Listing which has been submitted electronically in .XML format and is hereby incorporated by reference in its entirety. Said .XML copy, created on Mar. 21, 2025, is named “T2849-US_SL.xml” and is 10,000 bytes in size. The sequence listing contained in this XML file is part of the specification and is hereby incorporated by reference herein in its entirety.

### FIELD OF THE INVENTION

The present invention relates to a liquid kidney biopsy that can be used for non-invasive and early diagnosis of IgA glycosyl abnormalities.

### BACKGROUND OF THE INVENTION

IgA nephropathy (IgAN) is the most common glomerular disease worldwide, with an estimated incidence of 2.5 cases per 100,000 adults per year. Approximately 40% of IgAN cases progress to end-stage kidney disease (ESKD) within 20 years. The average age of onset is 30 years, with clinical manifestations including persistent or episodic macro-hematuria, micro-hematuria, proteinuria, and systemic hypertension. The prevalence, presentation, progression, and clinical course of IgAN are highly variable, largely influenced by the age of disease onset.

Recent advances in the understanding of IgAN pathobiology have redefined it as an autoimmune disease. Genome-wide association studies (GWAS) have shown that dysregulation of both innate and adaptive immunity through various pathways leads to IgAN manifestations, indicating it encompasses a complex group of disease statuses involving multiple patterns of immune dysregulation.

Further, it was proposed that the multi-hit model of IgAN pathogenesis was involving: (1) A genetic defect in the glycosylation of the IgAQ1 hinge region leads to the secretion of polymeric Gd-IgAQ1 molecules, with an estimated heritability of 50-70%. Aberrant mucosal immune activation, particularly in the bowel, in response to exogenous antigens, stimulates Gd-IgAQ1 overproduction. (2) Gd-IgAQ1 induces the secretion of specific autoantibodies, possibly involving genetic predispositions and molecular mimicry of bacterial antigens. (3) Circulating IgG and Gd-IgAQ1 immune complexes are fundamental for renal damage, binding the soluble IgA Fc alpha receptor (CD89) and inducing mesangial proliferation via interactions with CD89, mesangial transferrin receptor 1, and transglutaminase 2. (4) These large immune complexes evade hepatic clearance and deposit in the mesangium, inducing mesangial cell proliferation and secretion of extracellular matrix and cytokines such as TNF-alpha and IL-6. Activation of the complement system's alternative and lectin pathways further contributes to glomerular inflammation and damage in IgAN.

On the other hand, human IgAQ1 contains a hinge region (HR) with 21 amino acids and nine potential O-glycosylation sites, where typically fewer than six serine or threonine residues are modified by O-linked glycans. O-glycosylation starts with the addition of N-acetylgalactosamine (GalNAc) followed by galactose (Gal), and both can be modified by sialic acid (SA). IgAN is linked to abnormal O-glycosylation in the IgA1 HR, resulting in increased levels of galactose-deficient IgAQ1 (Gd-IgA1). The exposed GalNAc in Gd-IgA1 HR forms large immune complexes with anti-glycan IgG antibodies. Abnormal IgAQ1 glycosylation and glycan-specific antibody levels are promising biomarkers for IgAN diagnosis.

Generally established clinical biomarkers for diagnosing and prognosing IgAN include proteinuria, lower eGFR at diagnosis, and hypertension. Proteinuria≥1 g/day identifies individuals at risk for disease progression who may benefit from immunosuppressive treatment. Nevertheless, while suggesting an immune activity, proteinuria also reflects glomerular hyperfiltration and downstream tubulo-interstitial chronic damage, which are unlikely to respond to immunosuppressive treatment, limiting its use as a biomarker of immune monitoring.

Currently diagnosis of IgAN necessitates a kidney biopsy, which reveals dominant or co-dominant IgAQ1 immune complex deposits in the mesangium of the glomerulus, often accompanied by C3, and occasionally IgG and IgM deposits. Pathological lesions observed under light microscopy can range from mild mesangial hypercellularity to diffuse inflammatory features, including endocapillary leukocyte proliferation and/or crescent formation. Crescentic IgA nephropathy, defined as crescents present in more than 50% of glomeruli, is exceedingly rare. In advanced stages, focal segmental glomerulosclerosis and global sclerosis are frequently observed.

Recent small-scale studies have evaluated repeat kidney biopsy to monitor responses to immunosuppressive treatment. Despite its high diagnostic and prognostic value for IgAN, kidney biopsy is invasive, associated with morbidity and discomfort, and unlikely to be repeated during the disease course. That is to say the diagnosis of IgA nephropathy requires a renal biopsy in combination with light microscopy, immunofluorescence and electron microscopy. Renal biopsy involves an invasive procedure and might cause complications such as internal hemorrhage. Thus, its use is typically limited to prognosis assessment at diagnosis.

Importantly, urine is an invaluable tool in medical diagnosis due to its non-invasive nature, making sample collection simple and painless. Urine tests are cost-effective, widely available, and can be easily performed in various healthcare settings. Additionally, they can offer early detection of diseases, enabling timely intervention and management.

In addition, urine samples offer several advantages over serum samples, including non-invasive and easier collection, lower risk of infection, higher patient compliance, cost-effectiveness, and suitability for frequent monitoring. Urine provides a rich source of biomarkers, requires less stringent storage conditions, and has a reduced risk of contamination, making it a superior option for many clinical applications.

Therefore, it is exceptionally urgent crucial to develop a urine-based IgAQ1 glycan analysis with high readability and specificity. This non-invasive approach would enhance the diagnosis of IgA nephropathy and facilitate the formulation of optimal follow-up treatment strategies.

### SUMMARY OF THE INVENTION

In view of the above-mentioned problem, the present invention provides a non-invasive diagnostic method for rapid diagnosing of IgAN patients.

Another aspect of the present invention provides a strategy to apply a liquid biopsy for monitoring an IgAQ1 complex in urinary sample from IgAN subjects using a monoclonal antibody or/and a lectin via immunoassay.

In one embodiment, the present invention also provides a method for unraveling the mechanisms of IgA nephropathy and establishing therapeutical strategies.

In an alternative embodiment, the instant invention also provides a method for detecting galactose-deficient IgAQ1 in a subject, comprising: (a) obtaining a urinary sample from a subject; and (b) using a monoclonal antibody or a lectin in an immunoassay to detect galactose-deficient IgAQ1 in the subject.

In certain embodiment, the present invention further provides a method of diagnosing IgA nephropathy in a subject, comprising: (a) using a monoclonal antibody or a lectin as probes to be applied in an immunoassay; and (b) determining whether the monoclonal antibody or the lectin binds to a galactose-deficient IgA1, binding of the monoclonal antibody or the lectin to the galactose-deficient IgAQ1 indicating the subject has or is at risk of developing IgA nephropathy.

In one embodiment, the lectin is Crenomytilus grayanus lectin (CGL).

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

Detailed description of the invention is given in the following embodiments with reference to the accompanying drawing.

### BRIEF DESCRIPTION OF THE DRAWINGS

The drawings described herein are for illustration purposes only and are not intended to limit the scope of the present teachings in any way.

FIG. 1 shows EASON2 heavy chain peptide sequence (SEQ ID NO:2) and it's CDRs are underlined.

FIG. 2 shows EASON2 light chain peptide sequence (SEQ ID NO:3) and CDRs are underlined.

FIG. 3 is a schematic diagram of CGL binding assay in urine of IgAN via ELISA-based analysis. CGL, Crenomytilus grayanus lectin; IgAN, IgA nephropathy; HC, health control subjects; DN, diabetic nephropathy; SLE, systemic lupus erythematosus. **p<0.01; ***p<0.005; ***p<0.001.

FIG. 4 is a schematic diagram of mouse monoclonal against Gd-IgAQ1 antibody (EASON2) binding assay in urine of IgAN via ELISA-based analysis. IgAN, IgA nephropathy; HC, health control subjects; DN, diabetic nephropathy; SLE, systemic lupus erythematosus. ****p<0.001.

FIG. 5 is a schematic diagram of enhanced sensitivity and specificity for detecting urinary Gd-IgAQ1 using combined results based on ELISAs with CGL and EASON2 in IgA nephropathy patients.

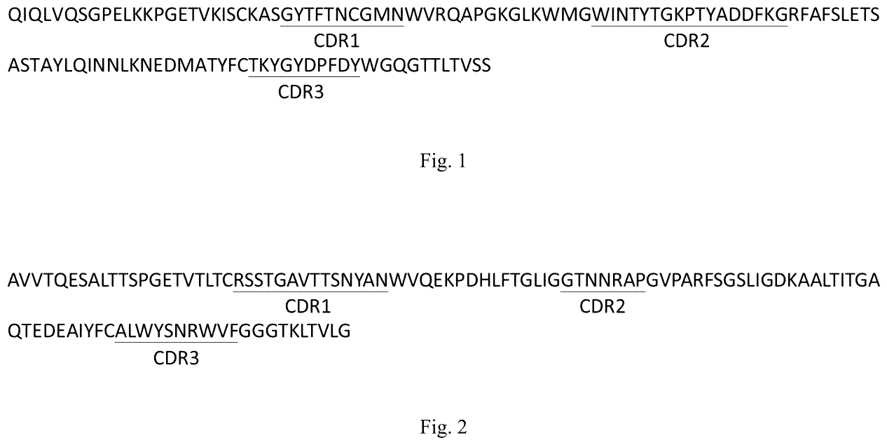

### DETAILED DESCRIPTION OF THE INVENTION

While preferred embodiments of the invention are shown and described herein, such embodiments are provided by way of example only and are not intended to otherwise limit the scope of the invention. Various alternatives to the described embodiments of the invention may be employed in practicing the invention.

Unless otherwise defined, all technical terms used herein have the same meaning as commonly understood by one of ordinary skill in the art to which this invention belongs.

In one embodiment, the lectin of the invention is Crenomytilus grayanus lectin (also called CGL).

Optionally, the CGL also can be produced by genetic engineering. A sequence coding CGL with His-tag is cloned into pET21a (+) vector, and then transformed into competent cells to express CGL in bacteria.

Sequences of His-tagged CGL (artificial CGL) (SEQ ID NO: 1) which can be employed in accordance with the invention are shown hereinbelow:

SEQ ID NO: 1:
MTTFLIKHKASGKFLHPYGGSSNPANNTKLVLHSDIHERMYFQFDVVDER
WGYIKHVASGKIVHPYGGQANPPNETNMVLHQDRHDRALFAMDFFNDNIM
HKGGKYIHPKGGSPNPPNNTETVIHGDKHAAMEFIFVSPKNKDKRVLVYA
HHHHHHHH

The CGL can bind specific sugars. The lectin of the invention is a dimer composed of a stable α-chain structure, and has three sites that bind sugars on each monomer. The CGL of the invention can be combined with galactose (Gal), N-acetylgalactosamine (GalNAc), and globular trisaccharides, and is classified as a family of galactose-binding lectin.

The CGL has a very high affinity to mucin-type glycoprotein, that characterized by the high content of the O-glycoside-bound chains, preferably has a high affinity to N-acetyl-2-deoxy-2-amino-galactose (GalNAc/Gal), particularly specifically binds to Galacotose-deficient IgAQ1 (Gd-IgA1). Therefore, the lectin of the invention can be used to develop a novel IgAQ1 galactose-deficient O-glycosylation assay for detecting the existence of Gd-IgAQ1 to determine a subject who is suffered from IgA nephropathy (IgAN) or not.

On the other hand, the term “O-linked sugar chain” means a structure in which a sugar chain is bound via an —OH group contained in each amino acid side chain of an amino acid residue of serine (Ser) or threonine (Thr) of a protein. Specific examples of the O-linked sugar chain include T antigen (TF antigen), sialyl T antigen, Tn antigen, sialyl-Tn antigen, and the like.

Examples of the amino acid residue of a polypeptide to which an O-linked sugar chain is bound include an amino acid residue of serine (Ser) or threonine (Thr) in an amino acid sequence of the hinge region of the IgAQ1 heavy chain polypeptide.

In the present invention, as the amino acid residue of the hinge region polypeptide to which the O-linked sugar chain on the IgAQ1 heavy chain polypeptide is bound, any of Ser or Thr residues in the amino acid sequence of the hinge region of IgA1 heavy chain polypeptide is available. Examples of these preferably include a sugar chain binding site comprising at least one amino acid residue selected from the group consisting of threonine at position 225, threonine at position 228, serine at position 230, serine at position 232 and threonine at position 236, in the amino acid sequence of human IgAQ1 heavy chain polypeptide.

The monoclonal antibody of the present invention has a binding activity to the sugar chain-deficient IgA1. The monoclonal antibody of the present invention includes, not limited, an antibody produced by a hybridoma. The anti-sugar chain-deficient IgAQ1 antibody can be obtained by culturing the hybridoma or administering the hybridoma cell into an animal to cause ascites tumor in the animal and separating and purifying the culture or the ascites.

The animal immunized with an antigen may be any animal, so long as a hybridoma can be prepared, and mouse, rat, hamster, rabbit or the like is suitably used.

The monoclonal antibody is an antibody secreted by a single clone of antibody-producing cells, and recognizes only one epitope (also called antigen determinant) and has the uniformity in amino acid sequence (primary structure).

Examples of the epitope include a single amino acid sequence, a three-dimensional structure consisting of an amino acid sequence, an amino acid sequence having a sugar chain bound thereto, a three-dimensional structure consisting of an amino acid sequence having a sugar chain bound thereto, and the like, which a monoclonal antibody recognizes and binds to. Examples of the epitope of the monoclonal antibody of the present invention include a three-dimensional structure of the sugar chain-deficient IgA1 protein.

Examples of the monoclonal antibody of the present invention include any monoclonal antibody, so long as it recognizes and also binds to the heavy chain hinge region of the sugar chain-deficient IgA1.

Examples of the monoclonal antibody which competes with the monoclonal antibody of the present invention include, specifically, a monoclonal antibody which has a competitive reaction for a variety of monoclonal antibodies and the epitope present in the heavy chain hinge region of the sugar chain-deficient IgA1, as described above.

The “heavy chain” of an antibody, used in the present specification, refers to a larger one of the two types of polypeptide chains present in all antibody molecules in a conformation present in nature. The “light chain” of an antibody used in the present specification refers to a smaller one of the two types of polypeptide chains present in all antibody molecules in a conformation present in nature.

Optionally, in an exemplary embodiment of the present invention, the monoclonal antibodies of the present invention include, but is not limited to, EASON2 heavy chain (SEQ ID NO:2), and EASON2 light chain (SEQ ID NO:3).

Sequences that can be employed in accordance with the above-mentioned embodiment are shown herein below:

SEQ ID NO: 2 (EASON2 heavy chain):
QIQLVQSGPELKKPGETVKISCKASGYTFTNCGMNWVRQAPGKGLKWMGW
INTYTGKPTYADDFKGRFAFSLETSASTAYLQINNLKNEDMATYFCTKYG
YDPFDYWGQGTTLTVSS
SEQ ID NO: 3 (EASON2 light chain):
AVVTQESALTTSPGETVTLTCRSSTGAVTTSNYANWVQEKPDHLFTGLIG
GTNNRAPGVPARFSGSLIGDKAALTITGAQTEDEAIYFCALWYSNRWVFG
GGTKLTVLG

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region.

Three complementarity determining regions (hereinafter referred to as “CDR”) in each of variable regions of the heavy chain and the light chain. The three CDRs are called CDR1, CDR2 and CDR3, counting from the amino terminus of the antibody chain. The monoclonal antibody of the present embodiment includes a CDR having the following amino acid sequence.

TABLE 1
Amino acid sequences of CDRs for EASON2 heavy chain

| SEQ ID No. | Description | Sequence |
|---|---|---|
| SEQ ID NO: 4 | CDR1 | GYTFTNCGMN |
| SEQ ID NO: 5 | CDR2 | WINTYTGKPTYADDFKG |
| SEQ ID NO: 6 | CDR3 | TKYGYDPFDY |

TABLE 2
Amino acid sequences of CDRs for EASON2
light chain

| SEQ ID No. | Description | Sequence |
|---|---|---|
| SEQ ID NO: 7 | CDR1 | RSSTGAVTTSNYAN |
| SEQ ID NO: 8 | CDR2 | GTNNRAP |
| SEQ ID NO: 9 | CDR3 | ALWYSNRWVF |

A subject may be a human being or a non-human animal, such as cat, dog, rabbit, cattle, horse, sheep, goat, monkey, mouse, rat, gerbil, guinea pig, pig, but is preferably a human.

Optionally, determining binding level to the galactose-deficient IgAQ1 comprises performing an assay from the group consisting of a Western blot, an enzyme-linked immunosorbent assay (ELISA), an immunoaffinity assay, a dot-blot assay and modified test tube binding analysis.

As used herein, the terms “antibody” or “antigen binding fragment thereof” include man-made antibodies such as monoclonal antibodies (mAbs) and/or an antigen binding fragments thereof, produced by conventional hybridoma technology, by phage display, and/or recombinant technology. The terms include both intact immunoglobulin molecules including, for example, a polyclonal antibody, a monoclonal antibody (mAb), a monospecific antibody, a bispecific antibody, a polyspecific antibody, as well as portions, fragments, regions, peptides and derivatives thereof (provided by any known technique, such as, but not limited to, enzymatic cleavage, peptide synthesis, or recombinant techniques), such as, for example, immunoglobulin devoid of light chains, Fab, Fab′, F(ab′) 2, Fv, scFv, antibody fragment, diabody, Fd, CDR regions, or any portion or peptide sequence of the antibody that is capable of binding antigen or epitope. The antibody, or antigen binding fragment thereof, may be a human antibody, a humanized antibody, an animal antibody (e.g. camelid antibody), or chimeric antibody. In one embodiment, the “antigen binding fragment thereof” is a single chain antibody, a single chain variable fragment (scFv), a Fab fragment, or a F(ab′) 2 fragment.

In one embodiment, the SEQ ID NO: 1-SEQ ID NO: 9 may be used to determine IgAN patients by ELISA-based binding assay. For example, the SEQ ID NO: 1-SEQ ID NO: 9 is labeled with a tag, including, but is not limited to, biotin, His tag, fluorescent substance (Cy3 or Cy5), or Digoxigenin (Dig). The tag-labeled lectin is mixed with a sample, and then the amount of Gd-IgAQ1 in the samples is analyzed by detecting an absorbance value or fluorescence intensity to determine a subject who is suffered from IgAN or not.

Further, the present invention also provided a diagnostic kit for detection of Gd-IgAQ1 in IgAN patients via a non-invasive manner.

In an alternative embodiment, the instant invention also provides a method for detecting galactose-deficient IgAQ1 in a subject, comprising: (a) obtaining a urinary sample from a subject; and (b) using a monoclonal antibody of EASON2 or a CGL as probes in an immunoassay to detect galactose-deficient IgAQ1 in the subject.

The monoclonal antibody of EASON2 or the CGL of the invention can be used to determine the existence of Gd-IgAQ1 in urinary sample because the monoclonal antibody of EASON2 or/and the CGL of the invention is capable of specifically binding to Gd-IgA1. If the level of Gd-IgAQ1 in the serum collected from a subject is higher than a specific value, the subject may be indicated as an IgAN patient. If the level of Gd-IgAQ1 in the serum collected from a subject is lower than a specific value, the subject is not an IgAN patient.

In summary, the present invention provides a novel rapid, effective and high-specificity method for non-invasively detecting Gd-IgAQ1 developed by the monoclonal antibody of EASON2 or/and the CGL. The monoclonal antibody of EASON2 or/and the CGL as probes of the present invention has huge potential for developing a rapid screening reagent or lateral flow immunochromatography (IFLA) test paper for commercial use. In other word, the present invention provides a novel diagnostic method for improving or replacing the invasive surgery acquiring the renal tissue sections to detect IgAN.

### EXAMPLES

The embodiments encompassed herein are now described with reference to the following examples. These examples are provided for the purpose of illustration only, and the disclosure encompassed herein should in no way be construed as being limited to these examples, but rather should be construed to encompass any and all variations which become evident as a result of the teachings provided herein.

Experimental data were analyzed with GraphPad Prism 7 (GraphPad Software, Inc., CA, USA) or SPSS version 22.0 (IBM, NY, USA). All data are presented as mean±standard error of mean (SEM). Student's t test (t test) was used in the testing of hypothesis for comparison of means between the groups. A p-value less than 0.05 (typically ≤0.05) is statistically significant. Further, ROC curve and logistic regression analyses were plotted by SPSS.

The present invention provides a novel approach to evaluate the synergistic effects of combining two diagnostic reagents, CGL and EASON2, in predicting IgAN. By utilizing binary logistic regression analysis, the method quantitatively assesses whether the combined use of these reagents results in a significant improvement in predictive performance compared to their individual use.

The process begins with the labeling of samples, where cases of IgAN are marked as 1 and healthy controls as 0. This binary classification is used as the dependent variable in the logistic regression model, with the reagent values serving as independent variables. ROC curve analysis is then performed to derive individual and combined predictive accuracies, which are graphically represented to identify the optimal cut-off points.

Upon identifying the synergistic effect, the invention enables the combination of the reagents' cut-off points to produce a composite ROC curve. The optimal cut-off from this curve is used to categorize observations, which are then subjected to further analysis via SPSS software, generating a cross-tabulation table. This table illustrates the effectiveness of the combined reagents in correctly classifying IgAN cases and healthy controls, thereby providing a robust method for evaluating the diagnostic synergy of the reagents.

### Example 1

### CGL Binding Assay in Urine of IgAN

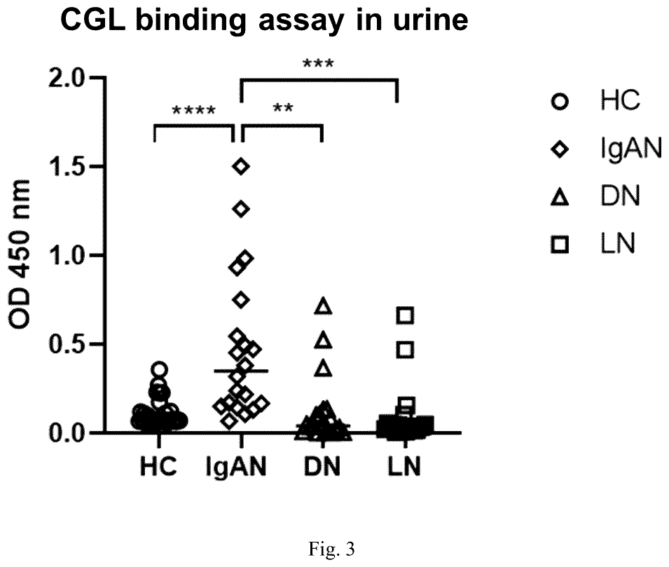

ELISA-based analysis. 96-well microplates were coated with CGL, then diluted urine were added, followed by incubating with biotin conjugated anti-human IgA antibody. Absorbance was measured. CGL was bound to urinary Gd-IgA1. The results showed that levels of Gd-IgA1 were significantly increased in the urine of IgAN patients, compared with those of in DN, SLE, and HC (FIG. 3).

### Example 2

### Mouse Monoclonal Against Gd-IgAQ1 Antibody (EASON2) Binding Assay in Urine of IgAN

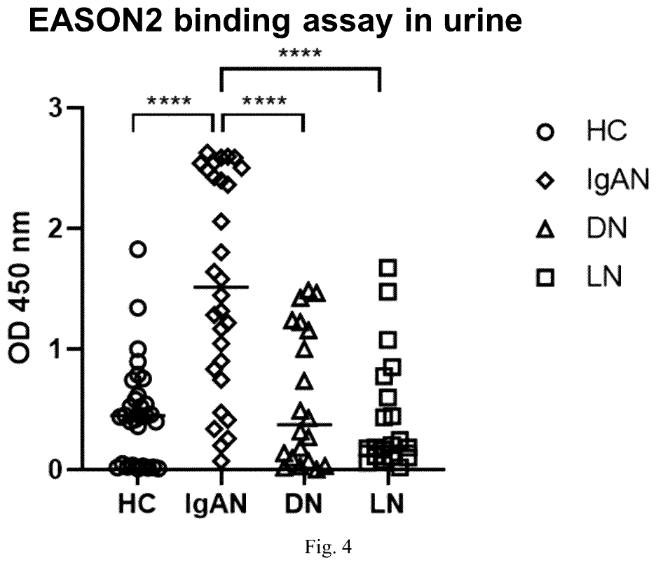

ELISA-based analysis. 96-well microplates were coated with mouse anti-human Gd-IgAQ1 antibody (EASON2), then diluted urine were added, followed by incubating with biotin conjugated anti-human IgA antibody. Absorbance was measured. EASON2 was bound to urinary Gd-IgA1. The results showed that levels of Gd-IgA1 were significantly increased in the urine of IgAN patients, compared with those of in DN, SLE, and HC (FIG. 4).

### Example 3

Enhanced Sensitivity and Specificity for Detecting Urinary Gd-IgAQ1 Using Combined-Based ELISAs with CGL and EASON2 in IgA Nephropathy Patients

Complementary character of CGL ELISA and EASON2 ELISA were evaluated by logistic regression analysis, that combined results based on ELISAs with CGL and EASON2 showed higher sensitivity and specificity for detecting urinary Gd-IgAQ1 in IgAN patients than those of CGL ELISA alone or EASON2 ELISA alone, respectively. The optimal cut-off point for CGL yields a sensitivity of 85% and the optimal cut-off point for EASON2 with a sensitivity of 75%. Among 20 urinary samples from IgA nephropathy patients, 17 tested positive using the CGL lectin ELISA (20×0.85=17), while 15 tested positive using the EASON2 mouse monoclonal antibody ELISA (20×0.75=15). The sensitivity of combined-based ELSIAs are 90%, and the AUC up to 0.851.

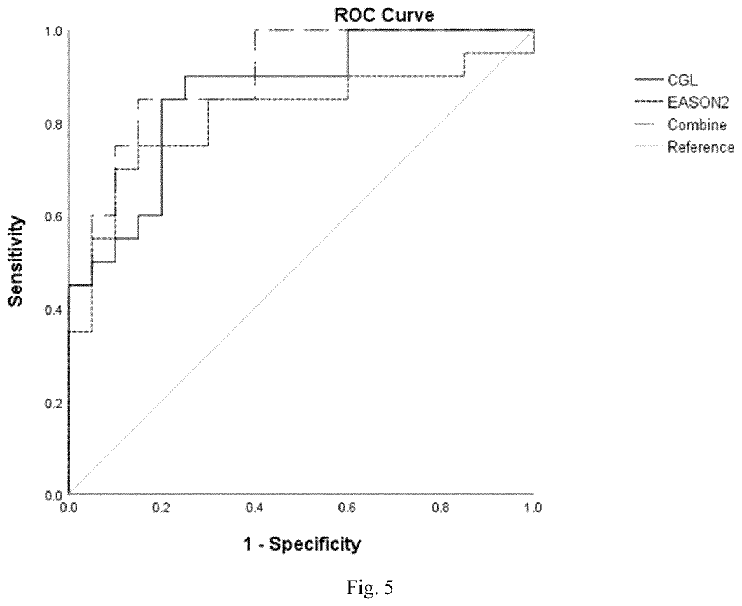

As an illustration, please refer to Table 3 and FIG. 5, IgAN patients and non-IgAN patients were divided into two groups according to the optimal cut-off value of urinary Gd-IgA1, which was obtained by CGL-base ELISA or/and EASON2-base ELISA.

TABLE 3

| | | | Subject | | |
|---|---|---|---|---|---|
| | | | HC | IgAN | Total |
| CGL & | <cut-off | Count | 15 | 2 | 17 |
| EASON2 | value | % within subjects | 75.0% | 10.0% | 42.5% |
| | >=cut-off | Count | 5 | 18 | 23 |
| | value | % within subjects | 25.0% | 90.0% | 57.5% |
| Total | | Count | 20 | 20 | 40 |
| | | % within subjects | 100.0% | 100% | 100.0% |

The foregoing is illustrative of the present invention, and is not to be construed as limiting thereof. The invention is defined by the following claims, with equivalents of the claims to be included therein.

What is claimed is:

1. A method for detecting galactose-deficient O-glycosyl IgA1, comprising:
(a) obtaining a urinary sample from a subject; and
(b) using a first probe in a first immunoassay to detect galactose deficient O-glycosyl IgAQ1 in the subject;
wherein the probe is a CGL,
wherein the CGL set forth in SEQ ID NO:1;
(c) using a second probe in a second immunoassay to detect galactose deficient O-glycosyl IgAQ1 in the subject;
wherein the second probe is an IgG monoclonal antibody,
wherein the IgG monoclonal antibody is selected from SEQ ID NO: 2, and SEQ ID NO: 3.

2. A method according to claim 1, wherein an amino acid sequence in the heavy chain
(SEQ ID NO: 2) comprising the following:
(SEQ ID NO: 4)
GYTFTNCGMN;
(SEQ ID NO: 5)
WINTYTGKPTYADDFKG;
and
(SEQ ID NO: 6)
TKYGYDPFDY;
and

3. A method according to claim 2, wherein the subject is selected from the group consisting of IgA nephropathy (IgAN) subjects, systemic lupus erythematosus (SLE) subjects, diabetic nephropathy and healthy subjects.

4. A method according to claim 3, wherein the method is calculated by logistic regression.

5. A method for diagnosing a urinary sample from a subject with galactose-deficient O-glycosyl IgA1, comprising:
(a) using a first probe in a first immunoassay; and
(b) determining whether the first probe binds to galactose-deficient O-glycosyl of IgA1, the binding of the first probe to galactose-deficient O-glycosyl of IgAQ1 indicating that the subject has or is at risk of developing galactose-deficient O-glycosyl of IgA1;
wherein the first probe is a CGL,
wherein the CGL set forth in SEQ ID NO: 1
(c) using a second probe in a second immunoassay; and
(d) determining whether the second probe binds to galactose-deficient O-glycosyl of IgA1, the binding of the second probe to galactose-deficient O-glycosyl of IgAQ1 indicating that the subject has or is at risk of developing galactose-deficient O-glycosyl of IgA1;
wherein the second probe is an IgG monoclonal antibody,
wherein the IgG monoclonal antibody is selected from SEQ ID NO: 2, and SEQ ID NO: 3.

6. A method according to claim 5, wherein an amino acid sequence in the heavy chain
(SEQ ID NO: 2) comprising the following:
(SEQ ID NO: 4)
GYTFTNCGMN;
(SEQ ID NO: 5)
WINTYTGKPTYADDFKG;
and
(SEQ ID NO: 6)
TKYGYDPFDY;
and

7. A method according to claim 6, wherein the subject is selected from the group consisting of IgA nephropathy (IgAN) subjects, systemic lupus erythematosus (SLE) subjects, diabetic nephropathy and healthy subjects.

8. A method according to claim 7, wherein the method is calculated by logistic regression.

9. A method for detecting or diagnosing galactose-deficient O-glycosyl IgA1, comprising:
(a) obtaining a urinary sample from a subject; and
(b) using a probe in an immunoassay for determining whether the probe binds to galactose-deficient O-glycosyl of IgA1, the binding of the probe to galactose-deficient O-glycosyl of IgAQ1 indicating that the subject has or is at risk of developing galactose-deficient O-glycosyl of IgA1;
wherein the probe is a CGL or an IgG monoclonal antibody,
wherein the CGL set forth in SEQ ID NO:1,
wherein the IgG monoclonal antibody is selected from SEQ ID NO: 2, and SEQ ID NO: 3.

10. A method according to claim 9, wherein an amino acid sequence in the heavy chain
(SEQ ID NO: 2) comprising the following:
(SEQ ID NO: 4)
GYTFTNCGMN;
(SEQ ID NO: 5)
WINTYTGKPTYADDFKG;
and
(SEQ ID NO: 6)
TKYGYDPFDY;
and

11. A method according to claim 10, wherein the subject is selected from the group consisting of IgA nephropathy (IgAN) subjects, systemic lupus erythematosus (SLE) subjects, diabetic nephropathy and healthy subjects.

12. A method according to claim 11, wherein the method is calculated by logistic regression analysis.


SEQUENCE LISTING (9 sequences)

SEQ ID NO: 1 - protein, 158 aa, synthetic construct
MTTFLIKHKASGKFLHPYGGSSNPANNTKLVLHSDIHERMYFQFDVVDERWGYIKHVASGKIVHPYGGQANPPNETNMVLHQDRHDRALFAMDFFNDNIMHKGGKYIHPKGGSPNPPNNTETVIHGDKHAAMEFIFVSPKNKDKRVLVYAHHHHHHHH

SEQ ID NO: 2 - protein, 117 aa, synthetic construct
QIQLVQSGPELKKPGETVKISCKASGYTFTNCGMNWVRQAPGKGLKWMGWINTYTGKPTYADDFKGRFAFSLETSASTAYLQINNLKNEDMATYFCTKYGYDPFDYWGQGTTLTVSS

SEQ ID NO: 3 - protein, 109 aa, synthetic construct
AVVTQESALTTSPGETVTLTCRSSTGAVTTSNYANWVQEKPDHLFTGLIGGTNNRAPGVPARFSGSLIGDKAALTITGAQTEDEAIYFCALWYSNRWVFGGGTKLTVLG

SEQ ID NO: 4 - protein, 10 aa, synthetic construct
GYTFTNCGMN

SEQ ID NO: 5 - protein, 17 aa, synthetic construct
WINTYTGKPTYADDFKG

SEQ ID NO: 6 - protein, 10 aa, synthetic construct
TKYGYDPFDY

SEQ ID NO: 7 - protein, 14 aa, synthetic construct
RSSTGAVTTSNYAN

SEQ ID NO: 8 - protein, 7 aa, synthetic construct
GTNNRAP

SEQ ID NO: 9 - protein, 10 aa, synthetic construct
ALWYSNRWVF